(19) United States
(12) Patent Application Publication
(10) Pub. No.: US 2005/0045296 A1
(43) Pub. Date: Mar. 3, 2005

(54) Stabilized absorbent composite material and method for making

(76) Inventors: Gabriel Hammam Adam, Alpharetta, GA (US); Leon Eugene Chambers JR., Cumming, GA (US); Michael A. Daley, Appleton, WI (US); Eric Edward Lennon, Roswell, GA (US); Sridhar Ranganathan, Suwanee, GA (US)

Correspondence Address: KIMBERLY-CLARK WORLDWIDE, INC., 401 NORTH LAKE STREET, NEENAH, WI, 54956, US

(21) Appl. No.: 10/652,778

(22) Filed: Aug. 29, 2003

(51) Int. Cl. D21F 1/00; D21H 15/00; D21H 25/00; A61F 13/15; A61F 13/20

(52) U.S. Cl. 162/157.6; 162/100; 162/157.1; 162/157.7; 604/385.23; 604/385.01; 604/385.08; 604/374; 604/358

(57) ABSTRACT

Disclosed herein is a densified stabilized absorbent composite material and a method for making densified stabilized absorbent composite material which may be used in or as an absorbent core material for absorbent products such as personal care absorbent products.

### TECHNICAL FIELD

This invention relates to fibrous web materials generally and particularly to stabilized absorbent composite materials and a method for making the materials.

### BACKGROUND OF THE INVENTION

Disposable absorbent products such as mortuary, veterinary and personal care absorbent articles such as diapers, training pants, disposable swimwear, incontinence garments and pads, sanitary napkins, wipes and the like often include one or more layers of fibrous web materials, especially liquid absorbent fibrous web materials, as an absorbent core material, and a backing layer or moisture barrier layer which is impervious to fluid. Personal care absorbent products typically also include a surface for contacting the body of the user, and the absorbent fibrous web material is generally disposed between the body-contacting surface and the moisture barrier layer so that body fluids are absorbed into the product and are contained by the moisture barrier.

Such absorbent fibrous web materials are frequently formed as nonwoven fibrous webs, such as for example, a pulp fluff/superabsorbent composite structure which may also contain longer synthetic staple fibers, such as thermoplastic binder fibers. In order to improve the structural integrity when utilized as, or as part of, an absorbent core material, it is beneficial to bond or otherwise stabilize the structure of the fibrous web material. Methods are known in the art for providing structural integrity to absorbent fibrous web material such as adhesive bonding or, where thermoplastic binder fibers are employed, by the application of heat such as by through-air bonding in an oven with heated air.

It is often highly desirable to have such absorbent composite materials produced in a highly densified state, for ease of storage and transportation when the materials are stored in roll goods form. Additionally, highly densified absorbent composite materials are easier to handle in product converting operations wherein the materials are incorporated into the product as the absorbent core and result in more aesthetically pleasing “thin” personal care absorbent products such as thinner diapers or thin feminine care pad products. However, conventional methods for forming densified absorbent composite materials include heat activation of thermoplastic binder followed by densification, or simultaneous heat activation and densification, both of which can damage the structure of the absorbent composite material and/or its components, particularly the structure of superabsorbent materials, which can deleteriously affect fluid absorption characteristics of the material. Thus, there remain continuing opportunities for improved stabilized absorbent composite materials and methods for making stabilized absorbent composite materials.

### SUMMARY OF THE INVENTION

The present invention provides for a method of making a stabilized absorbent composite material including the steps of providing cellulosic fibers, thermoplastic bicomponent binder fibers and superabsorbent material, entraining in a stream of air as a mixture the cellulosic fibers, binder fibers and superabsorbent material, depositing the mixture on a moving forming surface to form an airlaid composite material, densifying the material, and then heating the airlaid composite to activate the binder fibers to stabilize the absorbent composite material. The superabsorbent material in the stabilized absorbent composite material may desirably be superabsorbent particles having a mass median particle size which is greater than about 80 percent, and more desirably at least about 90 percent, of the mass median particle size of those in a similar but non-densified material. In certain embodiments, the density of the stabilized absorbent composite material may be at least about 0.10 g/cm³, and for other embodiments the density may desirably be at least about 0.15 g/cm³ or at least about 0.20 g/cm³. The stabilized absorbent composite material may have a third insult fluid intake rate greater than about 3 ml/s, and desirably greater than about 4 ml/s. The method may further include the step of bonding one or more sheet layers to the stabilized absorbent composite material to form a laminate material, and the sheet layer or layers may desirably be such as a nonwoven sheet layer, a foam layer, a tissue layer or a thermoplastic film layer.

The invention further provides a densified stabilized absorbent composite material comprising cellulosic fibers, thermoplastic bicomponent binder fibers and superabsorbent particles, the stabilized absorbent composite material having a density of at least about 0.10 g/cm³, and wherein the superabsorbent particles have a particle size distribution defined by the weight percent of particles in each of a series of sized cuts. For the stabilized absorbent composite material, the weight percent of superabsorbent particles in the top two of the series of cuts is at least about 50 percent of the weight percent of superabsorbent particles in the top two cuts of a similar but non-densified material. In certain embodiments, the density of the stabilized absorbent composite material is desirably at least about 0.15 g/cm³. In another embodiment, the weight percent of superabsorbent particles in the top two of the series of cuts is at least about 70 percent of the weight percent of superabsorbent particles in the top two cuts of a similar but non-densified material. The stabilized absorbent composite material may further comprise one or more additional sheet layers bonded to it, and the sheet layer or layers may desirably be such as a nonwoven sheet layer, a foam layer, a tissue layer or a thermoplastic film layer. The stabilized absorbent composite material may desirably be used in or as a component of various personal care absorbent products.

### BRIEF DESCRIPTION OF THE DRAWING

FIG. 1 is a schematic illustration of an exemplary process line for making stabilized absorbent composite materials in accordance with the invention.

FIG. 2 is a schematic illustration of another embodiment of an exemplary process for making stabilized absorbent composite material.

FIG. 3 is a perspective view of a disposable diaper comprising the stabilized absorbent composite material of the invention.

FIG. 4 is an exploded perspective view of a testing apparatus used to measure fluid intake characteristics of absorbent composites.

### DEFINITIONS

As used herein and in the claims, the term “comprising” is inclusive or open-ended and does not exclude additional unrecited elements, compositional components, or method steps.

Accordingly, the term “comprising” encompasses the more restrictive terms “consisting essentially of” and “consisting of”.

As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and random symmetries.

As used herein the term “monocomponent” fiber refers to a fiber formed from one or more extruders using only one polymer. This is not meant to exclude fibers formed from one polymer to which small amounts of additives have been added for color, anti-static properties, lubrication, hydrophilicity, etc. These additives, e.g. titanium dioxide for color, are generally present in an amount less than 5 weight percent and more typically about 2 weight percent.

As used herein the term “conjugate fibers” refers to fibers which have been formed from at least two polymers, or the same polymer with different properties, extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement or an “islands-in-the-sea” arrangement. Conjugate fibers are taught in U.S. Pat. No. 5,108,820 to Kaneko et al., U.S. Pat. No. 5,336,552 to Strack et al., and U.S. Pat. No. 5,382,400 to Pike et al. For two component fibers, the polymers may be present in ratios of 75125, 50/50, 25/75 or any other desired ratios.

As used herein the term “biconstituent fibers” refers to fibers which have been formed from at least two polymers extruded from the same extruder as a blend. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner.

As used herein the term “nonwoven fibrous web” means a web having a structure of individual fibers or filaments which are interlaid, but not in an identifiable manner as in a knitted or woven fabric. Nonwoven fabrics or webs have been formed from many processes such as for example, meltblowing processes, spunbonding processes, and dry-forming techniques such as carding and air-laying processes. The basis weight of nonwoven fabrics is usually expressed in grams of material per square meter (gsm) or ounces of material per square yard (osy) and the fiber diameters useful are usually expressed in microns. (Note that to convert from osy to gsm, multiply osy by 33.91.)

As used herein, an “airformed” or “airlaid” web is a fibrous web structure formed primarily by a process involving deposition of loose, air-entrained fibers onto a porous forming surface. Generally the web comprises cellulosic fibers having typical lengths ranging from about 1 to about 5 millimeters (mm) such as those from fluff pulp that have been separated from a mat or “board” of pulp fibers, such as by a hammermilling process, and then entrained in a moving stream of air and deposited or collected on the porous forming surface. Longer cellulosic fibers may be used as well. There may also be other longer fibers such as synthetic staple fibers or thermoplastic binder fibers present, and typically following collection of the fibers on the forming surface the web is bonded by such means as thermal bonding or adhesive bonding and/or densified. Further, an airlaid web to which binder material is subsequently added can be considered within the scope of the term “airlaid” according to the present invention. In addition, superabsorbent materials in particulate or fiber form may be included in airlaid webs where desired. Equipment for producing airlaid webs includes the Rando-Weber air-former machine available from Rando Machine Corporation of Macedon, N.Y. and the Dan-Web rotary screen air-former machine available from Dan-Web Forming International A/S of Risskov, Denmark.

As used herein, the term “cellulosic” is meant to include materials having cellulose as a major constituent, and specifically comprising at least about 50 percent by weight cellulose or a cellulose derivative. Therefore the term cellulosic includes, without limitation, cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose.

As used herein, the term “hydrophilic” with regard to polymeric or cellulosic material means that the material has a surface free energy such that the material is wettable by an aqueous medium, i.e., a liquid medium of which water is a major component. The hydrophilicity of materials can be measured, for example, in accordance with the ASTM-D-724-89 contact angle testing procedure. For example, a hydrophilic polymeric material has an initial contact angle equal to or less than 90°. Depending on material application needs and degree of hydrophilicity desired, this term includes materials where the initial contact angle may desirably be equal to or less than about 75°, or even equal to or less than about 50°. The term “initial contact angle” as used herein indicates a contact angle measurement made within about 5 seconds of the application of water drops on a test film specimen. The term “hydrophobic” includes those materials that are not hydrophilic as defined. It will be recognized that hydrophobic materials may be treated internally or externally with surfactants and the like to render them hydrophilic, and that slightly or moderately hydrophilic materials may be treated to make them more hydrophilic.

### DESCRIPTION OF THE INVENTION

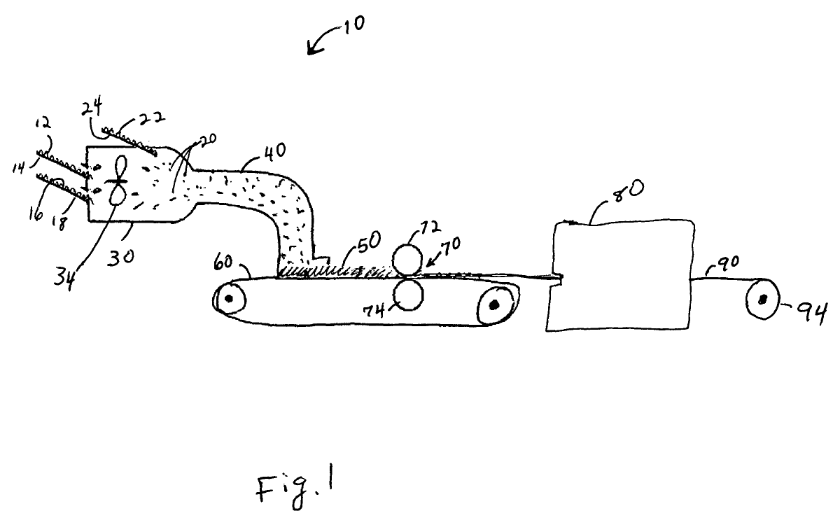

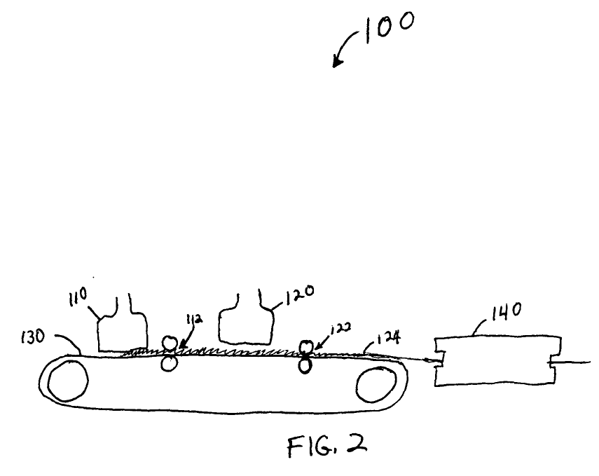

The present invention is directed to stabilized absorbent composite materials and methods for making stabilized absorbent composite materials. The invention will be described with reference to certain embodiments and with reference to an illustrated process line in FIG. 1 and FIG. 2. It will be apparent to those skilled in the art that these embodiments do not represent the full scope of the invention which is broadly applicable in the form of variations and equivalents as may be embraced by the claims appended hereto. It is intended that the scope of the claims extend to all such variations and equivalents.

The method of making the stabilized absorbent composite material comprises providing loose cellulosic fibers, thermoplastic binder fibers and superabsorbent materials and entraining these components into a moving airstream. The components are then collected on a moving forming surface to form a composite airlaid fibrous web material, which is densified and then undergoes heat activation of the binder to form the stabilized absorbent composite material. The fibrous web forming method may utilize conventional airlaying processes as are known in the art, such as is disclosed for example in U.S. Pat. No. 4,640,810 to Laursen et al., U.S. Pat. No. 4,494,278 to Kroyer et al., U.S. Pat. No. 5,527,171 to Soerensen and U.S. Pat. No. 4,375,448 to Appel et al., all incorporated herein by reference. The stabilized absorbent composite material comprises by weight from about 2 percent to about 20 percent thermoplastic binder fiber, from about 30 percent to about 80 percent superabsorbent material such as for example particulate superabsorbent material, and from about 18 percent to about 68 percent of cellulosic fibers. More particularly, the stabilized absorbent composite material comprises by weight from about 2 percent to about 10 percent thermoplastic binder fiber, from about 40 percent to about 80 percent superabsorbent particles, and from about 18 percent to about 58 percent of cellulosic fibers.

The thermoplastic binder fibers help to “stabilize” the material, i.e., help keep it from fragmenting or breaking apart under applied pressure, which is an especial hazard of pulp or cellulosic structures when wet. Suitable thermoplastic binder fibers include thermoplastic monocomponent and multicomponent fibers. Particularly suited are bicomponent thermoplastic binder fibers having one polymer component which melts at a temperature lower than the melting temperature of the second polymer component. Melting or partial melting of at least one polymer component of the bicomponent fiber allows the bicomponent fibers to form a tacky skeletal structure, which upon cooling, captures and binds or bonds to many of the pulp fibers. Additionally, the thermoplastic binder fibers may bond to each other and/or particles of superabsorbent material. Typically, the polymers of the bicomponent fibers are made up of different thermoplastic materials, such as for example, polyolefin/polyester (sheath/core) bicomponent fibers whereby the polyolefin, e.g. polyethylene sheath, melts at a temperature lower than the core, e.g., polyester. An example of a commercially available useful bicomponent thermoplastic binder fiber are the “CELBOND” fibers produced by KOSA Inc. (formerly Trevira Inc. and formerly Hoechst Celanese Corporation), Salisbury, N.C., under the designation T-255, which is a sheath-and-core fiber having a polyethylene sheath and a polyester core.

Typical thermoplastic and other polymers useful in fiber forming include polyolefins, e.g. polyethylene, polypropylene, polybutylene, and copolymers thereof, polyesters, e.g. polyethylene terephthalate, polyvinyl acetate, polyvinyl chloride acetate, polyvinyl butyral, acrylic resins, e.g. polyacrylate, and polymethylacrylate, polymethylmethacrylate, polyamides, namely nylon, polyvinyl chloride, polyvinylidene chloride, polystyrene, polyvinyl alcohol, polyurethanes, cellulosic resins, namely cellulosic nitrate, cellulosic acetate, cellulosic acetate butyrate, ethyl cellulose, etc., copolymers of any of the above materials, e.g. ethylene-vinyl acetate copolymers, ethylene-acrylic acid copolymers, styrene-butadiene block copolymers, and the like.

Suitable superabsorbent materials useful in the present invention include superabsorbent particles as are known in the art. Generally, superabsorbent materials include water-swellable, generally water insoluble materials capable of absorbing at least about 10 times their weight in water. More specifically, superabsorbent materials are capable of absorbing as much as 20 times their weight in water, and more specifically 50 times, 100 times, or even up to 300 times or more their weight in water (or other dispersion medium). Superabsorbent materials may be formed from organic material which may include natural materials such as agar, pectin, and guar gum, as well as synthetic materials such as synthetic hydrogel polymers. Synthetic hydrogel polymers include, for example, carboxymethylcellulose, alkali metal salts of polyacrylic acid and its copolymers, polyacrylamides, polyvinyl alcohol, ethylene maleic anhydride copolymers, polyvinyl ethers, hydroxypropylcellulose, hydroxypropyl acrylate, polyvinyl morpholinone, polymers and copolymers of vinyl sulfonic acid, polyacrylates, polyacrylamides, polyvinyl pyridine, and the like. Other suitable polymers include hydrolyzed acrylonitrile grafted starch, acrylic acid grafted starch, and isobutylene maleic anhydride copolymers and mixtures thereof. The hydrogel polymers are suitably lightly crosslinked to render the material substantially water insoluble. Crosslinking may, for example, be by irradiation or by covalent, ionic, Van der Waals, or hydrogen bonding. Suitable materials are available from various commercial vendors, such as the Dow Chemical Company (Midland, Mich.), Stockhausen Inc. (Greensboro, N.C.), and BASF (Charlotte, N. C.). The noncellulosic, synthetic hydrogel polymers are suitable for use in the present invention. Exemplary commercially available superabsorbent materials include the synthetic hydrogel polymer superabsorbent particles such as are available from Stockhausen Incorporated (Greensboro, N.C.) under the name “FAVOR” and designated “SXM-9543”.

Suitable cellulosic fibers for use in the invention include conventional pulp fibers as are well known in the industry such as thermomechanical pulp fibers, chemithermomechanical pulp fibers, chemimechanical pulp fibers, refiner mechanical pulp (RMP) fibers, stone groundwood (SGW) pulp fibers, and peroxide mechanical pulp (PMP) fibers. Suitable cellulosic pulp fibers commercially available include those designated “NB-416” available from the Weyerhaeuser Corporation (Federal Way, Wash.) and “Caressa 1300” available from Buckeye Technologies, Inc. (Memphis, Tenn.).

As shown in the exemplary process line 10 shown in side view in FIG. 1, a mat of cellulosic fibers 12 is fed down fiber chute 14 into air-laying apparatus 30. Thermoplastic binder fibers 16 are fed into airlaying apparatus 30 via fiber chute 18. Fan 34 draws in clumps of cellulosic fibers and thermoplastic binder fibers and then acts to individualize the fibers into a mixture of loose fibers 20 and entrain the loose fibers 20 in a moving airstream. Superabsorbent materials (“SAM”) such as SAM particles 22 are fed into the process via chute 24. It should be noted that although chute 24 is shown on the process line downstream (later in process) from fan 34, the SAM particles may also be added prior to fan 34. The mixture of loose fibers 20 and SAM particles 22 is then conducted along duct 40, until being collected as composite airlaid fibrous web material 50 upon the moving forming surface 60, which is a foraminous screen or porous belt or the like. Web formation may be assisted by a vacuum (not shown) positioned beneath forming surface 60.

Following web formation on forming surface 60, the composite airlaid fibrous web material is compacted or densified at nip 70 defined by rollers 72 and 74. Desirably, the bulk density of the composite airlaid fibrous web material following densification is at least about 0.10 g/cm³ and more desirably at least about 0.15 g/cm³. In certain embodiments, the bulk density is desirably even higher, as high as about 0.20 g/cm³ or 0.30 g/cm³, or higher. Rollers 72 and 74 defining nip 70 may be any rollers useful for compacting or densifying webs as are known in the art, such as for example steel rollers, rubber or plastic covered steel rollers, rubber rollers, or combinations thereof. Rollers 72 and 74 defining nip 70 may be heated or unheated but are desirably operated unheated or at ambient temperature. Where rollers 72 and 74 are heated they should be operated at a temperature less than the softening point of the thermoplastic binder fibers.

While the specific means for compacting or densifying the composite airlaid fibrous web material is not crucial, it is important that the material be compacted to or substantially to its final desired density prior to heat activation of the thermoplastic binder, and that the material not undergo further compaction or densification after heat activation. As is discussed below with reference to the Examples and Comparative materials, densification of the stabilized absorbent composite material following heat activation of the thermoplastic binder material produces deleterious effects on the final stabilized absorbent composite material's components, such as by grinding or crushing the SAM particles, and negatively affects the material's fluid handling ability such as the material's ability to rapidly intake fluid insults. Returning to FIG. 1, once the composite airlaid fibrous web material 50 has been compacted to the desired final bulk density, the thermoplastic binder fibers are activated by a heat activation step such as by passing the composite airlaid fibrous web material 50 through oven 80. Oven 80 provides heated air to soften or partially melt the thermoplastic binder fibers 16 or at least one component of the thermoplastic binder fibers where bicomponent fibers are used. The partially melted thermoplastic binder fibers 16 then act to bind or stabilize the composite airlaid fibrous web material by bonding to cellulosic fibers 12 and/or other thermoplastic binder fibers 16. Thermoplastic binder fibers 16 may also bond to SAM particles 22. Following heat activation of the thermoplastic binder fibers 16 in oven 80, the stabilized absorbent composite material 90 is wound up on roll 94 for storage or transport to product or material converting operations. Alternatively, instead of being wound up on roll 94 the stabilized absorbent composite material 90 may be sent directly to various product or other converting operations or for further processing.

The stabilized absorbent composite material may desirably range in basis weight from about 50 grams per square meter (gsm) or less up to about 1000 gsm, or higher. More particularly, the composite airlaid fibrous web material may desirably range in basis weight from about 200 gsm to about 800 gsm. In order to produce materials with basis weights in the heavier ends of these ranges, it may be useful or desirable to employ an airlaying process substantially as described with respect to FIG. 1 except utilizing multiple forming heads as is depicted in FIG. 2. In FIG. 2, the airlaying process line generally denoted 100 utilizes two airforming heads which sequentially deposit layers of the composite airlaid fibrous web material upon the forming surface. Upstream airforming head 110 and downstream airforming head 120 deposit the composite airlaid fibrous web material 124 onto forming surface 130. That is, upstream forming head 110 deposits web material onto forming surface 130 and then downstream airforming head 120 deposits additional web material onto the web material which is deposited by airforming head 110. The composite airlaid fibrous web material is compacted at nips 112 and 122 after being deposited by forming heads 110 and 120, respectively. Following compaction to final density at nip 122, the composite airlaid fibrous web material 124 is passed through oven 140 to heat activate the thermoplastic binder fibers and the stabilized absorbent composite material may be rolled up for storage or transport as was described with reference to FIG. 1, or may instead be directed to product converting applications.

It should be noted that although FIG. 2 shows two forming heads, process variations including three or more airforming heads may also be utilized. In addition, it should be noted that for certain applications it will be desirable to have a stabilized absorbent composite material which is substantially homogeneous throughout its depth, in which case all the forming heads will be supplied with substantially the same compositional mix of the various components (cellulosic fibers, binder fibers, SAM particles and/or additives). For other applications it may be desirable to have a stabilized absorbent composite material which is striated or layered throughout its depth, that is, having layers with differing compositions, so that a stabilized absorbent composite material can be produced having layers with different fluid handling and storage characteristics. For example, it may be desirable to have a stabilized absorbent composite material having one layer having a higher weight percentage of SAM particles than the other layer or layers. Other examples are possible, for example it may be desirable to have a stabilized absorbent composite material wherein one or more layers do not have any SAM particles. Also, although process line 100 is shown with a compaction nip after each forming head, it should be noted that it is possible to run with only a single compaction nip, following the final downstream forming head, which compacts or densifies the web material to its final desired density. However, having compaction nips after each forming head as is shown in FIG. 2 may be advantageous where it is desired to give some integrity to earlier-deposited layer or layers of web material, to protect them from disruption when the subsequent layers of the web material are deposited. In addition, depending on the final desired density, it may be advantageous to utilize both nips 112 and 122 and further to use one or more additional compaction nips prior to heat activation of the binder. In addition, the use of multiple nips allows for the ability to densify the various layers differentially, for example the first-deposited layer may have a greater density than subsequently deposited layers.

Depending on the desired end use for the nonwoven material, it may be beneficial to add other treatments or additives to the web such as for example treatments to impart or increase hydrophilicity, or colorants or pigments. Such additives or treatments may be incorporated in the stabilized absorbent composite material by introducing them into the airlaying process at some point prior to the collection of the composite airlaid fibrous web upon the forming surface, or may be added later in the process by spraying or otherwise depositing them on the web material. Other alternatives are also possible, and various additional potential processing and/or finishing steps known in the art such as aperturing, slitting, treating, or lamination of the fibrous web material with films, foams, nonwoven web layers or tissue layers, may be performed without departing from the spirit and scope of the invention.

As a few examples of the foregoing, the stabilized absorbent composite material may be combined into a laminate material that contains at least one layer of the stabilized absorbent composite material and at least one additional layer such as a woven or nonwoven fabric, a tissue, a film or a foam. The additional layer for the laminate may be selected to impart additional and/or complementary properties, such as additional liquid handling characteristics, liquid barrier and/or microbe barrier properties. The layers of the laminate can be bonded to form a unitary structure by a bonding process known in the art to be suitable for laminate structures, such as thermal, ultrasonic or adhesive bonding processes.

For example, the stabilized absorbent composite material may be laminated with one or more additional nonwoven sheet layers such as spunbond or meltblown layers, or combinations thereof such as the exemplary laminate structure disclosed in U.S. Pat. No. 4,041,203 to Brock et al., incorporated herein in its entirety by reference, which discloses a pattern bonded laminate of at least one fibrous nonwoven web, e.g., spunbond fiber web, and at least one microfiber nonwoven web, e.g., meltblown web. Alternatively, a breathable film can be laminated to the stabilized absorbent composite material to provide a breathable barrier laminate material useful as, for example, a backsheet material for personal care absorbent products. As yet another alternative, the stabilized absorbent composite material can be laminated to a non-breathable film to provide a high liquid barrier, or laminated to one or more foam type materials to provide resiliency and/or additional absorbency.

The stabilized absorbent composite material and/or laminates of the stabilized absorbent composite material with other layers is highly suitable for use as an absorbent layer or absorbent core layer in a personal care absorbent product. Personal care absorbent articles include such as diapers, training pants, disposable swimwear, incontinence garments and pads, sanitary napkins, wipes and the like.

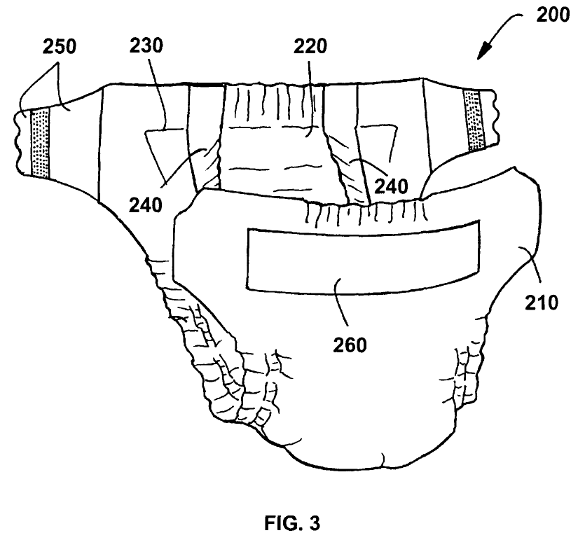

Turning to FIG. 3 there is shown an exemplary personal care article such as the diaper 200. Diaper 200, as is typical for most personal care absorbent articles, includes a liquid permeable body side liner 220, i.e., a body-facing or inner side, and a liquid impermeable outer cover 210, i.e., a non-body facing or outer side. Various woven or nonwoven fabrics can be used for body side liner 220 such as a spunbond nonwoven web of polyolefin fibers, or a bonded carded web of natural and/or synthetic fibers. Outer cover 210 is formed of a thin liquid barrier material such as for example a spunbond-meltblown layer, spunbond-meltblown-spunbond layer, or a thermoplastic polymer film layer. A polymer film outer cover may be embossed and/or matte finished to provide a more aesthetically pleasing appearance, or may be a laminate formed of a thermoplastic film and a woven or nonwoven fabric to provide a more aesthetically pleasing feel and sound or more “cloth-like” characteristics. Outer cover 210 may optionally be composed of a “breathable” material that is permeable to vapors or gas yet substantially impermeable to liquid, such as is known in the art. Examples of outer cover materials include but are not limited to those disclosed in U.S. Pat. No. 6,309,736 to McCormack et al., incorporated herein by reference in its entirety.

Disposed between liner 220 and outer cover 210 is an absorbent core 230 which comprises a layer of the stabilized absorbent composite material of the invention. Diaper 200 may further include optional containment flaps 240 made from or attached to body side liner 220. Suitable constructions and arrangements for such containment flaps are described, for example, in U.S. Pat. No. 4,704,116 to Enloe, incorporated herein by reference in its entirety. Still further, the diaper 200 can optionally include additional elements known to those skilled in the art, including but not limited to, elasticized leg cuffs, elastic waist band, and so forth.

To secure the diaper 200 about the wearer, the diaper will have some type of fastening means attached thereto. As shown in FIG. 3, the fastening means is a hook and loop fastening system including hook elements 250 attached to the inner and/or outer surface of outer cover 210 in the back waistband region of diaper 200 and one or more loop elements or patches 260 attached to the outer surface of outer cover 210 in the front waistband region of diaper 200. The loop material for loop patch 260 can be a woven, nonwoven or knitted loop material and may be secured to outer cover 210 of diaper 200 by known attachment means, including but not limited to adhesives, thermal bonding, ultrasonic bonding, or a combination of such means. As an alternative embodiment, a nonwoven loop material may cover all of, or substantially all of, the outer surface of outer cover 210.

### EXAMPLE

The Example stabilized absorbent composite material was produced substantially in accordance with the process described herein and Comparative stabilized absorbent composite material was produced for purposes of comparison. The Example and Comparative materials were produced using a 2-head airlaid forming line using Dan-Web forming heads to airlay the component fibers and materials. The SAM particles were added to the forming heads using two “Christy Coat-O-Matic Model 20 inch-DI-S” particle feeders available from the Christy Machine Company of Freemont, Ohio which were placed above each Dan-Web forming head substantially in accordance with the teachings of co-assigned U.S. Pat. App. No. 10/036854 to Chambers et al. (U.S. Application Publication No. 20030116890, Jun. 26, 2003).

Both the Example and Comparative materials contained by weight percentage approximately 46 percent Buckeye Technologies Caressa 1300 pulp, 50 percent Stockhausen SXM-9543 superabsorbent particles and 4 percent KoSA T-255 binder fibers, and were produced at an undensified or pre-compacted density of approximately 0.09 g/cm³ and a basis weight of approximately 660 gsm. Following deposition, the Example material was densified using two sets of unheated steel-on-rubber compaction rolls located just downstream of the second forming head. The compaction nip pressure was approximately 350 pounds per linear inch or PLI (about 63 kg/linear cm) and the Example material had a final density of about 0.18 g/cm³. The Example material was then passed through an air-impingement oven (air temperature approximately 350° F. (178° C.) and air face velocity about 400 ft/min (about 2 m/s)) to activate the binder fibers. The Comparative material was passed through the oven in its undensified state to activate the binder fibers (air temperature again approximately 350° F. (178° C.) and air face velocity about 400 ft/min (about 2 m/s)), and was then densified to a final density of about 0.18 g/cm³. To densify the Comparative material a set of steel-on-steel compaction rolls downstream of the oven was used. In order to densify the Comparative material to the same density as the Example material it was found that a pressure of approximately 830 PLI (about 148 kg/linear cm) was required. While not wishing to be bound by theory, we believe the increased pressure required to density the Comparative materials may be the result of a certain amount of resiliency added to the composite due to prior heat activation of the binder fibers.

Density of the materials was calculated by measuring the thickness or caliper of the material and the length, width and weight of the sample and calculating the density in units of grams per cubic centimeter. Thickness of a 4 inch by 4 inch (10.16 by 10.16 cm) sample of material was measured under a 3 inch (7.62 cm) diameter circular platen under a 0.2 PSI (1.38 kpa) load.

The Example and Comparative materials were tested for SAM particle size distribution and fluid intake rate as is described below. In addition, a similar but non-densified absorbent composite material was tested for SAM particle distribution. By the term “similar” what is meant is an absorbent composite material made under essentially the same process conditions and having essentially the same components except for the conditions or components mentioned. Here, the similar but non-densified absorbent composite material is the same material as either the Example or Comparative material at the point in the production process where material is deposited on the forming surface, but the similar but non-densified absorbent composite material does not undergo any densification step, either before or after heat activation of the binder material.

SAM Particle Size Distribution:

The SAM particle size distribution was measured for the Example and Comparative absorbent composite materials. The SAM particle size distribution was also measured for similar but non-densified absorbent composite material (i.e., after being airlaid into the composite material and deposited on the forming surface and undergoing heat activation of the binder material in the same way as the Example and Comparative materials but otherwise in a non-densified state) as a way to determine the initial or as-deposited particle size distribution. The particle size distributions were measured in a series of sized cuts using a stack of sieves over a pan with each lower sieve having smaller sized openings than the preceding sieve, and then measuring the weight of SAM particles retained in each sieve and on the pan. The sieves used had 850, 600, 300, 90 and 45 micron openings. The particle size distribution percent by weight was then calculated using the weight retained by each sieve and the pan divided by the initial total weight of SAM particles tested.

To perform the test, a 25 gram sample of the SAM particles is obtained from each of the materials by carefully pulling the material apart and separating out the SAM particles. The 25 gram sample of SAM is placed into the sieve stack by placing it into the sieve with the largest openings (850 microns). The sieve stack is then shaken for 10 minutes with a Ro-Tap mechanical Sieve Shaker, Model B available from W.S. Tyler, Inc. (Mentor, Ohio), or other similar shaking device. After shaking, the weight of SAM particles retained in each cut (retained by each sieve and by the bottom pan) is measured and recorded. The principle is that a particle retained in the top cut, i.e., retained by the sieve having 850 micron openings, is considered to have a particle size greater than 850 microns. A particle passing through that sieve but being retained by the sieve having 600 micron openings is considered to have a particle size greater than 600 microns but less than or equal to 850 microns. Similarly, particles passing through the sieve having 600 micron openings but being retained by the sieve having 300 micron openings are considered to have a particle size greater than 300 microns but less than or equal to 600 microns, etc. Finally, particles passing through all of the 850, 600, 300, 90 and 45 micron openings sieves to be retained on the pan are considered to have a particle size of 45 microns or less.

TABLE 1 lists the results of particle size distribution testing by listing the weight percentage of particles in each cut for each of the non-densified, Example and Comparative materials. Sample size for each particle size distribution test was 25 grams of SAM particles.

TABLE 1

| | | 850 → | 600 → | 300 → | 90 → | |
|---|---|---|---|---|---|---|
| | >850 | 600 | 300 | 90 | 45 | 45-0 |
| Material | microns | microns | microns | microns | microns | microns |
| Non-densified | 1.12 | 19.75 | 49.70 | 28.31 | 0.63 | 0.49 |
| Example | 0.30 | 17.52 | 54.22 | 26.84 | 0.60 | 0.52 |
| Comparative | 0.06 | 8.78 | 46.95 | 40.35 | 1.96 | 1.90 |

As can be seen from TABLE 1, the weight percentage of SAM particles greater than 600 microns is about 21 percent for the similar but non-densified material. This percentage represents the combined weight percent of particles in the top two cuts, where the top two cuts are the 850 and 600 micron cuts. After densification and heat activation/stabilization of the Example material, the combined weight percent of particles in the top two cuts is still about 18 percent. Stated another way, the Example stabilized absorbent composite material still had over 85 percent of the top two cuts SAM particle weight compared to the similar but non-densified material. However, after heat activation and then densification for the Comparative material, the combined weight percent of particles in the top two cuts drops to less than 9 percent. Therefore, the Comparative stabilized absorbent composite material only had about 42 percent of the top two cuts SAM particle weight compared to the similar but non-densified material. Furthermore, the weight percentage of particles in the 300 microns and smaller category (the bottom three cuts) increases from about 29 percent by weight in the similar but non-densified material to about 44 percent by weight for the Comparative material. However, for the Example material the bottom three cuts are still only about 28 percent by weight of the SAM particles.

In addition, a mass median particle size can be calculated from the particle size distribution data in TABLE 1. The mass median particle size is determined by plotting the cumulative particle size distribution curve (i.e., the total mass percentage of material not passing through a particular sieve vs. size of that sieve opening) and by interpolating the sieve opening which corresponds to 50 percent of the cumulative distribution. That is, the particle size where an equal amount of the total mass of particles is contained in particle sizes higher and lower than the mass median particle size. A linear interpolation can be done by identifying the two cuts that bracket 50 percent of the cumulative distribution and identifying the particle size that would correspond to 50 percent. For example, in the similar but non-densified material 20.87 percent of the total mass of particles is above 600 microns while 70.57 percent of the total mass of particles is above 300 microns. Therefore, the mass median particle size (MMPS) for the similar but non-densified material is calculated as:
MMPS (non-densified)=600−(600−300)*(50−20.87)/(70.57−20.87)=424 microns.

The MMPS is calculated similarly for the Example and Comparative materials:
MMPS (Example)=600−(600−300)*(50−17.82)/(72.04−17.82)=422 microns.
MMPS (Comparative)=600−(600−300)*(50−8.84)/(55.79−8.84)=337 microns.

TABLE 2 shows the cumulative particle size distributions for each of the similar but non-densified, Example and Comparative Materials. TABLE 2 also lists the MMPS for each material as was calculated above.

TABLE 2

| | >850 | >600 | >300 | >90 | >45 | >0 | Mass |
|---|---|---|---|---|---|---|---|
| Material | microns | microns | microns | microns | microns | microns | Median |
| Non-densified | 1.12 | 20.87 | 70.57 | 98.88 | 99.51 | 100 | 424 |
| Example | 0.30 | 17.82 | 72.04 | 98.88 | 99.48 | 100 | 422 |
| Comparative | 0.06 | 8.84 | 55.79 | 96.14 | 98.10 | 100 | 337 |

As can be seen from TABLE 2, the mass median particle size for the Example material (422 microns) is over 99 percent of the mass median particle size for the similar but non-densified material (424 microns). However, for the Comparative material the mass median particle size drops to 337 microns, which is less than 80 percent of the mass median particle size for the similar but non-densified material. Therefore, it appears that the heat activate-then-densify process for the Comparative material is unduly destructive of the SAM particles, fracturing and breaking the SAM particles up into smaller fragments. While not wishing to be bound by theory, we believe this SAM particle destruction is caused by either the higher pressures required to densify the material after binder heat activation, or by the interaction of drying of the SAM particles (during binder heat activation) followed by the densification pressure, or both, which have a grinding and damaging effect on the SAM particles and may crack and/or break individual particles into smaller sized particles. This destructive grinding can have deleterious effects on the fluid handling abilities of the stabilized absorbent composite material, especially in the case of multiple or serial fluid insults, as for example when the stabilized absorbent composite material is used in or as part of an absorbent core material in a personal care absorbent product and is expected to take up and retain bodily fluid exudates.

Therefore it is beneficial to minimize SAM particle damage during processing and the stabilized absorbent composite material should have at least about 50 percent of the top two cuts SAM particle weight compared to the similar but non-densified material, and desirably this ratio may be 60 percent, 70 percent, 80 percent or even higher. It is also highly desirable that the mass median particle size or MMPS of the SAM particles in the stabilized absorbent composite material remain as close as possible to the MMPS of a similar but non-densified material. Desirably, the MMPS for the densified stabilized absorbent composite material will be higher than 80 percent of the MMPS for a similar but non-densified material, and more desirably will be 90 percent or more of the MMPS for a similar but non-densified material.

Fluid Intake Testing:

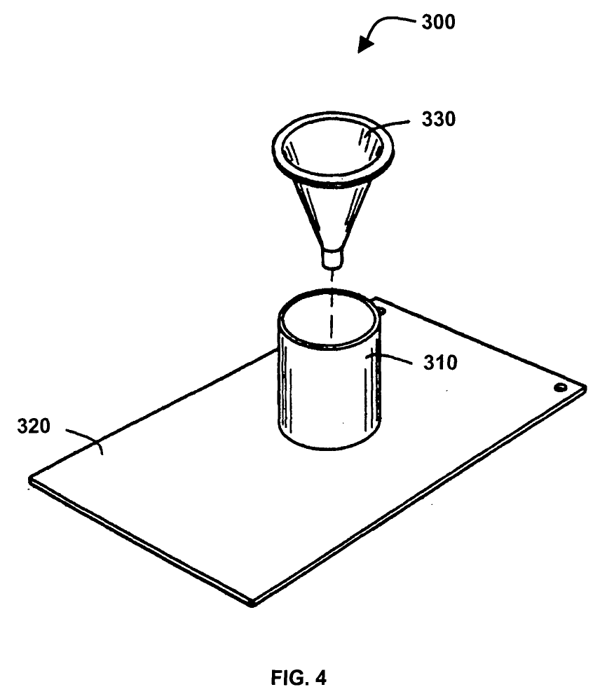

To simulate real-life fluid handling requirements and estimate potential leakage of the absorbent composite materials in-use in a personal care absorbent product, the rate of fluid intake upon a third insult was measured for the Example and Comparative materials. The third insult fluid intake was measured substantially in accordance with the fluid intake measurement portion of the “Fluid Intake and Flowback Evaluation” or “FIFE” test described in U.S. Pat. No. 5,147,343 to Kellenberger, incorporated herein by reference, with the modifications as described below. Generally, for the fluid intake portion of the test, the sample to be tested is placed flat and smooth under a 360 gram cylinder plate assembly such as is shown in FIG. 4. FIG. 4 depicts a cylinder plate assembly 300 having a cylinder 310 which extends to the bottom plane of the 7 inch by 7 inch (17.78 cm by 17.78 cm) plate 320. The sample to be tested (not shown) is placed flat and smooth under the cylinder and plate assembly such that the cylinder 310 is located over the desired portion of the sample to be tested. Under the sample to be tested is a raised platform (not shown) which is ½ inch high (1.27 cm) by 4 inch by 4 inch (10.16 cm by 10.16 cm). During testing, funnel 330 is placed inside and rests on top of cylinder 310. A specified amount of synthetic urine or normal saline is poured through the funnel 330 and thereby through cylinder 310. The time elapsing between the first fluid contact with the sample and the time when fluid disappears into the sample is measured with a stop watch. Fifteen minutes after the initial fluid insult is imbibed, a second insult of the same size is introduced. The time to imbibe the second fluid insult is measured as was done for the first insult. Finally, fifteen minutes after the second fluid insult is imbibed, a third fluid insult of the same size is introduced and the time to imbibe the fluid is again measured. For the Example and Comparative materials, the fluid intake measurement portion of the FIFE test was performed substantially as described above with the following modifications and characteristics. A 4 inch by 4 inch (10.16 cm by 10.16 cm) sample of material was used for each test, a 2.5 inch (6.35 cm) tall by 1 inch (2.54 cm) interior diameter cylinder was used, and for each insult 30 milliliters of normal saline were used. The time for the materials to absorb the third 30 milliliter insult was measured in seconds and the intake rate calculated in milliliters per second (ml/s) by dividing the 30 milliliters by the time in seconds. Each of the materials was tested 6 times and the results were averaged. The Example material performed at a rate of 4.5 ml per second in the third insult while the Comparative material third intake rate was less than 70 percent of the Example's fluid intake rate at about 2.9 ml per second.

It will be apparent to those skilled in the art that other alterations or modifications than those discussed herein may be made without departing from the spirit and scope of the present invention. It is therefore intended that all such modifications, alterations and other changes be encompassed by the claims. Numerous other patents have been referred to in the specification and to the extent there is any conflict or discrepancy between the teachings incorporated by reference and that of the present specification, the present specification shall control.

1. A method of making a stabilized absorbent composite material comprising:
a) providing cellulosic fibers;
b) providing thermoplastic bicomponent binder fibers;
c) providing superabsorbent material;
d) entraining in a stream of air as a mixture the cellulosic fibers, the bicomponent binder fibers and the superabsorbent material;
e) depositing the mixture on a moving forming surface to form an airlaid composite material;
f) densifying the airlaid composite material; and then
g) heating the airlaid composite to activate the bicomponent binder fibers to stabilize the absorbent composite material.

2. The method of claim 1 wherein the superabsorbent material in the stabilized absorbent composite material is a particulate superabsorbent having a mass median particle size, and wherein the mass median particle size is greater than about 80 percent of the mass median particle size of superabsorbent particles in a similar but non-densified material.

3. The method of claim 2 wherein the mass median particle size in the stabilized absorbent composite material is at least about 90 percent of the mass median particle size of superabsorbent particles in a similar but non-densified material.

4. The method of claim 1 wherein the density of the stabilized absorbent composite material is at least about 0.10 g/cm³.

5. The method of claim 4 wherein the density of the stabilized absorbent composite material is at least about 0.15 g/cm³.

6. The method of claim 5 wherein the density of the stabilized absorbent composite material is at least about 0.20 g/cm³.

7. The method of claim 4 wherein the stabilized absorbent composite material has a third insult fluid intake rate greater than about 3 ml/s.

8. The method of claim 7 wherein the stabilized absorbent composite material has a third insult fluid intake rate greater than about 4 ml/s.

9. The method of claim 2 further comprising the step of bonding at least one sheet layer to the stabilized absorbent composite material to form a laminate.

10. The method of claim 4 further comprising the step of bonding at least one sheet layer to the stabilized absorbent composite material to form a laminate.

11. The method of claim 10 wherein the at least one sheet layer is a nonwoven sheet layer, a foam layer, a tissue layer or a thermoplastic film layer.

12. A stabilized absorbent composite material formed in accordance with the method of claim 1.

13. A stabilized absorbent composite material formed in accordance with the method of claim 4.

14. A personal care absorbent product comprising the stabilized absorbent composite material of claim 13.

15. A densified stabilized absorbent composite material comprising cellulosic fibers, thermoplastic bicomponent binder fibers and superabsorbent particles, the stabilized absorbent composite material having a density of at least about 0.10 g/cm³, the superabsorbent particles having a particle size distribution defined by the weight percent of particles in each of a series of sized cuts, wherein the weight percent of superabsorbent particles in the top two of the series of cuts is at least about 50 percent of the weight percent of superabsorbent particles in the top two cuts of a similar but non-densified material.

16. The stabilized absorbent composite material of claim 15 wherein the density is at least about 0.15 g/cm³.

17. The stabilized absorbent composite material of claim 16 wherein the weight percent of superabsorbent particles in the top two of the series of cuts is at least about 70 percent of the weight percent of superabsorbent particles in the top two cuts of a similar but non-densified material.

18. The stabilized absorbent composite material of claim 16 further comprising at least one sheet layer bonded thereto.

19. The stabilized absorbent composite material of claim 18 wherein the at least one sheet layer bonded thereto comprises a tissue layer, a nonwoven layer, a foam layer or a film layer.

20. The stabilized absorbent composite material of claim 17 further comprising at least one sheet layer bonded thereto, wherein the at least one sheet layer comprises a tissue layer, a nonwoven layer, a foam layer or a film layer.

21. A personal care absorbent product comprising the stabilized absorbent composite material of claim 15.

22. A personal care absorbent product comprising the stabilized absorbent composite material of claim 18.

23. A personal care absorbent product comprising the stabilized absorbent composite material of claim 20.
